(19) United States
(12) Patent Application Publication
(10) Pub. No.: US 2006/0292217 A1
(43) Pub. Date: Dec. 28, 2006

(54) Nutritional supplement and soft gelatin capsule delivery system

(76) Inventors: Robbin D. Schmidt, Plainfield, IL (US); Marijo Clark, Valencia, CA (US); Fernando Salinas, Los Angeles, CA (US); David C. Madsen, Woodland Hills, CA (US); Jones Y.C. Chan, Valencia, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/447,337

(22) Filed: Jun. 5, 2006

Related U.S. Application Data
(60) Provisional application No. 60/687,414, filed on Jun. 3, 2005.

(51) Int. Cl. A61K 33/04; A61K 31/59; A61K 31/7076; A61K 9/64; A61K 31/22; A61K 31/202

(52) U.S. Cl. 424/456; 424/702; 514/167; 514/547; 514/560; 514/46

(57) ABSTRACT

A nutritional composition and method of introducing the composition into a soft gelatin capsule. The composition includes effective amounts of omega-3 fatty acid, vitamin D and selenium. The method includes forming each component into a unitary form, forming a soft gelatin capsule and introducing the unitary composition into the capsule. The soft gelatin capsule then serves as a delivery system for the composition when the capsule is introduced into the body of the user.

### RELATED APPLICATIONS

This application claims the benefit of the earlier filing date of U.S. Provisional Application Number 60/687,414, filed Jun. 3, 2005.

### BACKGROUND

1. Field

A nutritional composition and soft gelatin capsule for delivery of the composition to the body.

2. Background

The body is a complex system prone to many harmful substances that may weaken the systems of the body and cause numerous health problems. One line of defense against such harmful substances is the immune system. The immune system is a natural defense mechanism of the body responsible for fighting disease. The immune system acts to identify foreign substances in the body, and if harmful, fight off any inflammation or injury caused by the substances. The processes of the immune system are triggered by the body's natural inflammatory response, among other mechanisms. When harmful substances are introduced into the body, blood flow to the affected area is increased and the area experiences an influx of immune cells and secretions. In this way, the immune system is warned of the foreign substances and begins fighting off the infection.

It is normal for the body to continually express both pro-inflammatory and anti-inflammatory characteristics. A modem lifestyle, however, combined with sub-optimal diet, leads to a condition in which there is a mild (or more pronounced), but chronic or continual imbalance. Generally, the pro-inflammatory condition prevails in the case of a chronic imbalance in western societies.

Most medical methods for suppressing inflammation use drugs. There are many disadvantages to using drugs, including overdosing, side effects and costs. Additionally, many drugs target a single metabolic process and have one mechanism of action. Since each person's body is unique due to genomic differences, such a unilateral approach increases the potential for non-responsiveness. Thus, balancing the inflammatory response in a safe and effective way remains a problem.

Gelatin capsules are recognized carriers for certain vitamin, mineral and medicinal agents. The use of gelatin material in the manufacture of foods or capsules (e.g. vitamin capsules, supplement capsules, mineral capsules, etc.) has become popular, particularly as a human consumable, because of its digestibility and suitability as a carrier. In particular, the gelatin material is easily digested in the stomach and gelatin material is compatible with many vitamin, mineral and medicinal agents. In certain cases, in fact, gelatin material provides a better carrier in terms of stability of the active agents than other recognized methods of delivery to the body.

In forming a gelatin capsule containing an active agent, a gelatin mass (e.g., gelatin water and glycerin) is typically formed into a sheet. Such sheets are brought together with the active agent and individual capsules of the combination are singulated from the sheet. A particular dosage of the active ingredient may then be administered to the body when the user consumes the recommended number of capsules.

A safe and low cost mechanism for balancing inflammatory responses which targets many processes affecting inflammation remains desirable.

### BRIEF DESCRIPTION OF THE DRAWINGS

The following illustration is by way of example and not by way of limitation in the figures of the accompanying drawings in which like references indicate like elements. It should be noted that references to “an” or “one” embodiment in this disclosure are not necessarily to the same embodiment, and such references mean at least one.

FIG. 1 is a flow chart of one embodiment of a method for forming a supplement and soft gelatin capsule delivery system.

### DETAILED DESCRIPTION

A composition is disclosed. In one aspect, the composition is directed at balancing the inflammatory responses of the body by oral administration of the composition in a soft gelatin capsule or softgel form. In this context, the composition is a nutritional supplement including oil-based nutritional ingredients combined with mineral, vitamins and/or herbal components that are believed to effect heart health and inflammatory responses. It is believed that the combination of these components will have a synergistic effect on the inflammatory responses.

In one embodiment, the composition is made of a mixture of suitable active ingredients including, but not limited to a source of omega-3 fatty acids such as, fish oil; a source of vitamin D and a source of selenium. Omega-3 fatty acids have known antiarrhythmic, antithrombotic and anti-inflammatory properties. Examples of omega-3 fatty acids may include, but are not limited to, alpha-linolenic acid, eicosapentaeonoic acid (“EPA”), docosapentaenoic acid and docosahexaenoic acid (“DHA”). In one embodiment, the composition may include a fish oil concentrate. In an embodiment where a fish oil concentrate is used, the concentrate may have a specified concentration of omega-3 fatty acids. In one embodiment, the specified concentration of omega-3 fatty acids may be a combination of EPA and DHA in a specific ratio. In one embodiment, the omega-3 fatty acids may be derived from sources other than fish oil such as, but not limited to, vegetable oils (alpha-linolenic acid) such as chia seed (Salvia hispanica), walnut oil, hemp seed, soybean, pumpkin and black currant, and eggs.

Vitamin D is believed to help maintain a healthy immune system and regulate cell growth and differentiation within the body. Recognized forms of vitamin D include vitamin D2(ergocalciferol) and vitamin D3(cholecalciferol). In one embodiment, the composition may include vitamin D3. In one embodiment, the composition may include vitamin D2, vitamin D3 or a combination of both. In one embodiment, vitamin D3 may be derived from any commonly recognized source. In another embodiment, sources of vitamin D3 may include, for example, egg yolks, soybean oil, fish oils, fish liver and dairy products. In still another embodiment, vitamin D may be synthetically manufactured. Such manufacture may include chemically modified forms of Vitamin D3 (e.g. derivatives of Vitamin D). In one embodiment where vitamin D is synthetically manufactured, vitamin D3 and/or vitamin D2 may be present in a powder form. In another embodiment, the composition may include vitamin D2 solubilized in soybean oil. In one embodiment, the composition may include, but is not limited to, a combination of dry powder forms of vitamin D2 and/or vitamin D3.

Selenium is a nonmetallic element chemically related to oxygen and sulfur. The composition may include a suitable form of selenium. A suitable form of selenium may include, but is not limited to, a selenium yeast, a selenium dioxide (e.g., selenium dioxide tritrate), a sodium selenate, a sodium selenite or a selenomethionine.

In another embodiment, additional suitable active ingredients of the composition may include an antioxidant and/or a carotenoid. In one embodiment, a suitable antioxidant may include, but is not limited to, a polyphenol. Examples of a suitable polyphenol include, but are not limited to, grape seed extract, grape skin extract, green tea extract, strawberry extract, raspberry extract, bilberry extract, red wine powder and bioflavonoids. Examples of a suitable carotenoid may include, but are not limited to, beta-carotene, astaxanthin, lutein, xeanthin, vegetable-based and animal-based waxes. Both polyphenols and carotenoids have been shown to support a healthy immune system.

Additional active ingredients suitable for the composition may include, but are not limited to, methylsulfonyl methane (“MSM”), S-adenosyl methionine (“SAME”), turmeric, polymethoxylated flavones from orange oil, rose hip extract, glucosamine, chondrotin and antioxidants such as Vitamin C and Vitamin E.

The active ingredients of the composition may further be combined with a suitable excipient. A suitable excipient may include, but is not limited to, fuma silica, glyceryl monostearate, glyceryl behenate, hydrogenated vegetable oil, tocopheryolpolyethylenesuccinate (“TPGS”).

In another embodiment, the active ingredients of the composition may be combined with a suitable lubricant. In one embodiment, a suitable lubricant may include, but is not limited to, for example, lecithin, polysorbate 80 or another similar substance. In another embodiment, the active ingredients of the composition may be combined with a suitable suspending agent. In one embodiment, a suitable suspending agent may be, for example, yellow beeswax or another similar substance. In still another embodiment, the composition may include, but is not limited to, a mixture of active ingredients, a lubricant and/or a suspending agent.

In one embodiment, the composition may be administered as a supplement for a daily diet along with a meal. In another embodiment, the composition may be administered in oral dosage form. Suitable oral dosage forms may include, but are not limited to, liquid, powder, capsule, tablet, cereal and troche forms. In one embodiment, the composition in oral dosage form may be administered once a day along with a meal.

In one embodiment, the composition may include the active ingredients combined in amounts sufficient to achieve an effect on the inflammatory response of the body. In one embodiment, the composition may include the active ingredients in a dietary acceptable amount. In an embodiment where the composition is administered once a day, the active ingredients may be present in the amounts of approximately 2400 mg of fish oil, 1200 IU vitamin D3 and/or 100 micrograms (μg) of selenium. In another embodiment, the composition may include from about 150 mg to 3,630 mg of fish oil, from about 30 IU to 10,000 IU vitamin D3 and/or from about 10 μg to 200 μg of selenium.

In an embodiment where the composition includes fish oil, a suitable fish oil may include approximately 600 mg of omega-3 fatty acids. In another embodiment, fish oil may include approximately 720 mg or some range between 600 mg and 720 mg of omega-3 fatty acid. In another embodiment, the fish oil may include from about 37.5 mg to 907 mg of omega-3 fatty acids. In another embodiment, a concentrate of fish oil may be used. Where fish oil concentrate is used, a suitable concentrate may include EPA and DHA in a ratio of approximately 3:2 (e.g. 18% EPA to 12% DHA from mackerel fish oil).

In still another embodiment where the composition is administered once a day, the composition may include approximately 2400 mg of fish oil concentrate, 1200 IU vitamin D3, 100 μg of selenium and/or 50 mg of an antioxidant, such as, but not limited to, grape seed extract. In another embodiment, the composition may include, but is not limited to, from about 150 mg to 3,630 mg fish oil, from about 30 IU to 10,000 IU of vitamin D3, from about 10 μg to 200 μg of selenium dioxide and/or from about 25 mg to 788 mg of an antioxidant, such as, but not limited to, grape seed extract.

In an embodiment where the composition is administered once a day, the composition may further include approximately 0.83 percent of a lubricant of a surfactant, such as, but not limited to lecithin. The composition may further include approximately 3 percent of a suspending aid, such as, but not limited to, yellow beeswax. In another embodiment, the amount of lecithin may range from about 0.5 percent to 4 percent (w/w). In another embodiment, the amount of yellow beeswax may range from about 0.5 percent to about 15 percent by weight of the total composition (w/w).

In another embodiment, the composition in oral dosage form may be administered three times a day. In this embodiment, each dose of the composition may include approximately 800 mg of fish oil, 400 IU vitamin D3 and/or 33.3 μg of selenium. In another embodiment, each dose of the composition may include from about 50 mg to 1,210 mg of fish oil, from about 10 IU to 3,333 IU vitamin D3 and/or from about 3.3 μg to 66.6 μg of selenium. In still a further embodiment, the composition administered three times a day may include approximately 800 mg of fish oil, 400 IU vitamin D3, 33.3 μg of selenium and/or 50 mg of an antioxidant, such as, but not limited to, grape seed extract. In another embodiment, the composition may include from about 50 mg to 1,210 mg of fish oil, from about 10 IU to 3,333 IU vitamin D3, from about 3.3 μg to 66.6 μg of selenium and/or about 8.3 mg to 262.6 mg of an antioxidant, such as, but not limited to, grape seed extract.

In still another embodiment, the composition in an oral dosage form may include acceptable dietary amounts of one or more MSM, SAME, turmeric, polymethoxylated flavones from orange oil, rose hip extract, glucosamine, chondrotin, vitamin C and/or vitamin E. In another embodiment, the amounts of MSM, SAME, turmeric, polymethoxylated flavones from orange oil, rose hip extract, glucosamine, chondrotin, vitamin C and/or vitamin E may be any amount suitable to have the desired effect on the inflammatory response system.

A method is disclosed. In one aspect, the method is directed at forming the active ingredients into a unitary form and encapsulating the unitary form into the soft gelatin capsule.

In an embodiment where one of the active ingredients to be formed into a unitary form is fish oil, it is believed that the fish oil may provide the particular advantage of reducing the effective fill volume of the capsule. It is known that fish oils have specific gravity ranges from about 0.85 g/ml to 0.93 g/ml depending on their omega-3 fatty acid content. In one embodiment where fish oil and vitamin D3 are combined, the vitamin D3 is a high potency component that does not contribute to the overall specific gravity of the formulation. In one embodiment, where selenium dioxide (1% tritrate on dicalcium phosphate), grape seed extract, beeswax and lecithin are combined with the fish oil, the specific gravity of the fish oil is increased to 0.98 g/ml. The increased specific gravity provides an advantage when introducing the unitary form of the composition into the soft gelatin capsule in that it allows for a decrease in fill volume (expressed in minims). A decreased fill volume is desirable in that it allows for delivery of an effective dose of the composition in a lower volume amount. For example, a fish oil capsule at a fill weight of 0.927 g has a fill volume of 16.7 minims at a specific gravity of 0.90 g/ml. At the sample fill weight of 0.927 g, a composition described including from about 2400 mg of fish oil concentrate, 1200 IU vitamin D3, 100 μg of selenium and/or 50 mg of grape seed extract has a fill volume of 15.3 minims.

The following specific examples are set forth to illustrate the various methods for forming the active ingredients into a total composition or unitary form..

### EXAMPLE I

| Ingredient | Amount |
|---|---|
| Fish Oil | 2400 mg (600 mg omega-3 |
| | fatty acid) |
| Vitamin D3 | 1200 IU |
| Selenium dioxide | 100 μg |
| Grape Seed Extract (Standardized | 50 mg |
| for 95% polyphenols) | |
| Lecithin | 0.83% (w/w) |
| Yellow beeswax | 3.0% (w/w) |

In one embodiment, the unitary form of the composition may be formed using a one “pot” method with heat. A homogenization unit that combines, deaggregates and deaerates a fill material that may be put into a soft gelatin capsule is used to process the ingredients. In this embodiment, a suitable portion of the fish oil (e.g. about ½) is added to the homogenization unit. The fish oil is heated to above 40° C. and yellow beeswax is added. Heating and agitation continue until the fish oil and beeswax mixture clears. After the beeswax has completely melted, the vitamin D3 and lecithin are added. The temperature of the mixture is lowered to below 40° C. and selenium dioxide tritrate is added along with the grape seed extract. A homogenizer unit is activated and the mixture combined. The remainder of the fish oil is then added and the completed composition is combined for a specified time. The resulting composition exhibits little or no settling after 24 hours and has a specific gravity of approximately 0.98 g/ml. The average particle size of the solids within the mixture are less than 180 micrometers (μm). The mixture is tested for conformance to the master formulation and the target label claims.

### EXAMPLE II

| Ingredient | Amount |
|---|---|
| Fish Oil | 2400 mg (600 mg omega-3 |
| | fatty acid) |
| Vitamin D3 | 1200 IU |
| Selenium dioxide | 100 μg |
| Grape Seed Extract (Standardized for 95% | 50 mg |
| polyphenols) | |
| Lecithin | 0.83% (w/w) |
| Yellow beeswax | 3.0% (w/w) |

In one embodiment, the unitary form of the composition may be formed using a multi-step method with heat. In this embodiment, a portion of the fish oil is added to a jacketed mixing tank fitted with a suitably sized standard overhead mixer with a propeller type blade. While mixing, the fish oil is heated to above 40° C. and the beeswax is added. The fish oil and beeswax mixture are continuously mixed and heated until the beeswax is completely incorporated. Vitamin D3 and lecithin are then added and combined with further mixing. After adding vitamin D3 and lecithin, the mixture is cooled to below 40° C. and selenium dioxide tritrate and grape seed extract are added. The remainder of the fish oil is added and the completed formulation is mixed for a specified time. The fish oil mixture is transferred through a colloid mill and then into a film evaporator where the aggregate size is reduced to below 180 μm and all evidence of air is removed (deaerted), respectively. The resulting formulation exhibits little or no settling for 24 hours and has a specific gravity of approximately 0.98 g/ml. The mixture is tested for conformance to the master formulation and the target label claim.

### EXAMPLE III

| Ingredient | Amount |
|---|---|
| Fish Oil | 2400 mg (600 mg omega-3 |
| | fatty acid) |
| Vitamin D3 | 1200 IU |
| Selenium dioxide | 100 μg |
| Grape Seed Extract (Standardized for 95% | 50 mg |
| polyphenols) | |
| Lecithin | 0.83% (w/w) |
| Yellow beeswax | 3.0% (w/w) |

In one embodiment, the unitary form of the composition is formed by a one “pot” method without heat. In this embodiment, a portion of the needed fish oil is added to the homogenization unit. Fumed silica is added to the fish oil. Fumed silica may be purchased, for example from Degussa. After the fumed silica is completely incorporated, the vitamin D3 and lecithin are added. Then the selenium dioxide tritrate is added along with the grape seed extract. The homogenizer unit is activated and the mixture combined. The remainder of the fish oil is then added and the completed mixture combined for a specified time. The resulting formulation exhibits little or no settling after 24 hours and has a specific gravity of approximately 0.98 g/ml. The average particle size of the solids within the mixture is less than 180 μm. The mixture is tested for conformance to the master formulation and the target label claim.

### EXAMPLE IV

| Ingredient | Amount |
|---|---|
| Fish Oil | 2400 mg (600 mg omega-3 |
| | fatty acid) |
| Vitamin D3 | 1200 IU |
| Selenium dioxide | 100 μg |
| Grape Seed Extract (Standardized for 95% | 50 mg |
| polyphenols) | |
| Lecithin | 0.83% (w/w) |
| Yellow beeswax | 3.0% (w/w) |

In one embodiment, the unitary form of the composition is formed by a multi-step method without heat. In this embodiment, a portion of the fish oil is added to a jacketed mixing tank fitted with a suitably sized standard overhead mixer with a propeller type blade. While mixing, fumed silica is added to the fish oil. Mixing continues until all the fumed silica has been incorporated. Vitamin D3 and lecithin are then added and combined with further mixing. The selenium dioxide tritrate and grape seed extract are then added. The remainder of the fish oil is added and the completed formulation mixed for a specified time. The mixture is then transferred through a colloid mill and then into a film evaporator where the aggregate size is reduced below 180 μm and all evidence of air is removed (deaerated), respectively. The resulting formulation exhibits little or no settling for 24 hours and has a specific gravity of approximately 0.98 g/ml. The mixture is tested for conformance to the master formulation and the target label claim.

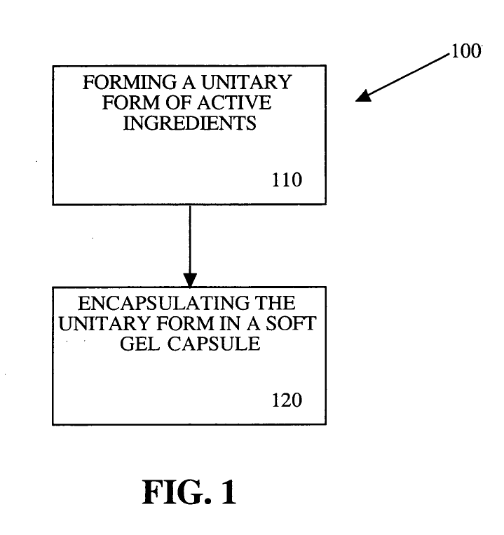

A method is further provided for forming the soft gelatin capsule and introducing the unitary form of the composition into the capsule (encapsulation process). FIG. 1 illustrates this method. In one embodiment, the method utilizes a rotary die encapsulator that either has casting drums parallel to a die/wedge zone or in series with the die/wedge zone. The encapsulation process brings a gel mass and the unitary form of the active ingredients together to form a soft gelatin capsule.

In one embodiment, the gel mass is cast onto two opposing chilled drums (either air or water cooled or a combination of both) forming a flexible gel ribbon with specific properties defined by the gel mass formulation. A unitary form of the composition may be formed as described above (block 110). The unitary form is placed into a positive displacement pump with feed lines that direct the unitary form into a heated injection wedge, which heats the gel ribbon prior to and during the encapsulation process. A matched set of counter-rotating dies with shaped cavities is located beneath the wedge. The gel ribbons are lubricated with appropriate oils and fed between the wedge and the dies. Heat is applied to the gel ribbon as it moves across the wedge. This unitary form is injected between the gel ribbons (block 120). As the injection through the wedge occurs, the injection expands the gel ribbon into the die cavity forming the capsule. Simultaneously the heated wedge softens and melts the gel ribbon to form the capsule's seam as the die applies pressure to further seal and cut the capsule from the gel ribbon. Typical encapsulation in-process controls are fill weight measurements and gel ribbon thickness.

Encapsulation parameters are set and verified to be within the established acceptable limits prior to the start of encapsulation. All product made during start up are rejected and disposed. Once the encapsulation parameters are within the adjust limits, the encapsulation is started. Encapsulation parameters such as gel ribbon thickness, wedge temperature, gel spreader box temperature, pump volume, and machine speed are continuously monitored during encapsulation. These parameters are or can be adjusted throughout the encapsulation run to assure conformance to the established in-process acceptance limits.

A set of in-process fill weights are taken at specified times. Typical times are every two (2) hours during encapsulation or dependent on the encapsulation batch size. Examples of in-process fill weight parameters are target weight and acceptance limit. Target weight is based on target potency and fill volume. Acceptance limit is typically ±3% solution, ±5% suspension of target fill weight.

A lubricant is applied to the top and bottom surfaces of the gel ribbon to ensure smooth movement of the gel ribbon over the machine's metal surfaces as well as providing a liquid bead seal at the wedge/gel ribbon interface, which is necessary for the exclusion of air in the capsule. The lubricant is typically food-grade, low viscosity oil, such as, but not limited to, Miglyol 810 or 812(fractionated coconut oil; mid-chain triglycerides) or Mineral Oil, light. A small amount of lubricant oil may be captured in the fill of each capsule, due to its presence on the inner surface of the gel ribbon that forms the capsule. The amount of lubricant contained within each capsule will vary depending on the capsule's size. Generally, there is less than two milligrams of lubricant per capsule. The lubricant oil is mechanically removed from the surface of the capsule during the tumble drying process using, for example, dry highly absorbent, lint-free towels that are introduced into the tumblers along with the formed capsules.

After the unitary form is introduced to the soft gelatin capsule, the wet soft gelatin capsules are conveyed either through an air handling system or a conventional mechanical conveyor belt into a capsule drying system. Representatively, the capsule drying system can consist of a sequential series of drying tumbler baskets (e.g., eight to 10 baskets) that are located in a controlled environment conducive to drying the soft gelatin capsules. The wet, freshly formed capsules enter a tumbler basket. The tumbler rotates continuously to provide air-flow over the capsule bed, thus promoting moisture removal from the capsule's gel shell. Tumbler air is made up of the controlled room air, which is maintained at 68°-72° F. and 20%-25% relative humidity. After a specified time, the tumbler basket empties into another basket, and the emptied basket is refilled with fresh product. The capsules continue to dry over time as they are moved down a sequence of baskets.

An alternative method of drying is to pass the freshly formed capsules through a secession of 5 rapidly rotating drums to drive off a bulk of the capsule's gel shell water content. The now firm capsules are then spread onto shallow trays and stacks of trays are assembled. These stacks are placed in environmentally controlled rooms or tunnels to continue the drying process. The drying process end point is achieved when the dried capsules have reached a hardness level not less than 8, as determined by a Bareiss hardness tester, or the gel shell moisture content is less than 9%. The capsules are then discharged and visually inspected for manufacturing artifacts, such as leakers, odd shapes and stuck togethers (clumps or clusters).

In another embodiment, modified release formulations of the composition are suitable. Representatively, a modified release formulation may be formed by applying an enteric coating to a soft gelatin capsule composition described above. An enteric coating on the composition may improve bioabsorption of the active ingredients by delaying breakdown of the capsule until the intestine rather than in the stomach. Other forms of modified release formulations are also suitable.

The above described compositions are suitable for administration as a nutritional or dietary supplement in connection with a method of reducing the harm associated with imbalances in a pro-inflammatory response. This includes, but is not limited to, methods of reducing the causes of coronary heart disease and improving cardiovascular health. The compositions are also suitable as a nutritional or dietary supplement to maintain a normal inflammatory balance. In one embodiment, the compositions may be taken as a dietary supplement in addition to meals.

In the preceding detailed description, specific embodiments are described. It will, however, be evident that various modifications and changes may be made thereto without departing from the broader spirit and scope of the claims. The specification and drawings are, accordingly, is to be regarded in an illustrative rather than restrictive sense.

What is claimed is:

1. A composition comprising dietary acceptable amounts of:
a source of omega-3 fatty acid;
a source of vitamin D or its derivative; and
a source selenium.

2. The composition according to claim 1, further comprising a dietary acceptable amount of an antioxidant wherein the antioxidant is a polyphenol.

3. The composition according to claim 2, further comprising dietary acceptable amounts of at least one of a carotenoid and a methylsulfonyl methane.

4. The composition according to claim 2, further comprising a dietary acceptable amount of S-adenosyl methionine.

5. The composition according to claim 1, further comprising at least one of a surfactant and a suspending aid.

6. The composition according to claim 5, wherein the surfactant is lecithin.

7. The composition according to claim 5, wherein the suspending aid is beeswax.

8. The composition according to claim 1, wherein the fish oil concentrate, source of vitamin D3 and source selenium are added in amounts suitable to effect an inflammatory response.

9. The composition of claim 1, wherein the source of omega-3 fatty acid is fish oil.

10. A method of making a composition comprising the active ingredients of omega-3 fatty acid, vitamin D and selenium into a soft gelatin capsule comprising the steps of:
forming the active ingredients into a unitary form; and
encapsulating the unitary form in a soft gelatin capsule.

11. The method of claim 10, wherein forming the active ingredients into a unitary form further comprises:
adding a dietary acceptable amount of at least one of an antioxidant, a carotenoid and a methylsulfonyl methane.

12. The method of claim 10, wherein the fill volume of the unitary form has a density greater than a density of an equivalent of any of the active ingredients.

13. The method of claim 10, wherein forming the active ingredients into a unitary form comprises:
mixing the active ingredients together;
heating the mixture of active ingredients;
mixing the active ingredients with at least one of a surfactant and a suspending aid; and
cooling the mixture.

14. The method of claim 13, wherein a portion of the fish oil concentrate is retained and added while the mixture is cooling.
